(19) United States
(12) Patent Application Publication
(10) Pub. No.: US 2026/0014363 A1
(43) Pub. Date: Jan. 15, 2026

(54) SELF-FLUSHING VALVE WITH COLLAPSIBLE VALVE MEMBER

(71) Applicant: CareFusion 303, Inc., San Diego, CA (US)

(72) Inventor: Christopher Jesse Zollinger, Chino, CA (US)

(21) Appl. No.: 18/767,815

(22) Filed: Jul. 9, 2024

(51) Int. Cl. A61M 39/10; A61M 39/00; A61M 39/24

(52) U.S. Cl. CPC A61M 39/10; A61M 39/24; A61M 2039/0009; A61M 2039/2426; A61M 2039/2433; A61M 2205/0216

(57) ABSTRACT

Connector assemblies are disclosed. The connector assembly includes a first housing having a fluid inlet and a second housing coupled to the first housing. The second housing includes a post defining an opening. The connector assembly includes a valve. The valve includes a channel disposed around the post to selectively isolate the opening of the fluid inlet. The channel defines a relief portion with a reduced valve body thickness. The valve is collapsible relative to the relief portion to permit fluid communication between the fluid inlet and the opening.

### TECHNICAL FIELD

The present disclosure relates generally to medical fluid valves and, more particularly, to valves modified to control medical fluid provided by syringes.

### BACKGROUND

For an intravenous (“IV”) application, valves, including needleless access valves, can be used to deliver a fluid to a patient. When a syringe provides sufficient pressure to a valve, the valve member collapses and permits downstream fluid delivery from the syringe to a catheter. However, as the syringe is removed from the valve, the valve member may not restore or return to an original position, compromising the operation of the valve. When these types of issues occur, the IV set needs to be replaced, leading to added costs and a negative patient experience.

### SUMMARY

Aspects of the present disclosure provide a connector assembly with a post (e.g., center post) and a valve that is positioned on the post. When engaged with a fluid delivery device (e.g., syringe), the valve is displaced (e.g., compressed) relative to the post, causing the post to protrude through the valve. The valve can include one or more features to allow the valve to collapse in an intentional, consistent, and/or even manner. The consistent collapse of the valve can minimize valve failures and prevent the valve from becoming stuck or folded over.

Additionally, as the fluid delivery device is removed from the connector assembly, the force provided by the fluid delivery devices reduces, allowing the valve to cover the post. Features of the valve can allow the valve to consistently restore or extend to an original sealing position. Accordingly, the valve promotes consistent and reliable operation and prevents valve failures or valve sticking, even during high pressure operations, such as power injections.

In accordance with at least some embodiments disclosed herein is the realization that that inconsistent collapse of valves can result in the valve becoming folded over or stuck, which can result in fluid leaks or unintended fluid administration, which can result in injury to a patient or a caregiver, such as by depriving the patient of a medicament, increasing the potential for infection to the patient, and exposing the caregiver to harmful medicaments. Additionally, a stuck or folded over valve can render the IV set unusable.

Accordingly, aspects of the present disclosure provide a connector assembly for use with a catheter, the connector assembly comprising a first housing comprising a fluid inlet, a second housing coupled with the first housing, the second housing comprising a post defining an opening, and a valve comprising a channel disposed around the post to selectively isolate the opening from the fluid inlet, the channel defining a relief portion having a reduced valve body thickness, wherein the valve is collapsible relative to the relief portion to permit fluid communication between the fluid inlet and the opening.

Some instances of the present disclosure provide a connector assembly for use with a catheter, the connector assembly comprising a first housing comprising a fluid inlet, a second housing coupled with the first housing, the second housing comprising a post defining an opening, and a valve comprising an exterior surface defining a dimple portion having an increased valve body thickness, the valve disposed around the post to selectively isolate the opening from the fluid inlet, wherein the valve is collapsible relative to the dimple portion to permit fluid communication between the fluid inlet and the opening.

Some instances of the present disclosure provide a collapsible valve for use with a connector, the valve comprising: a channel configured to selectively isolate to an opening of the connector; and an exterior surface defining a plurality of dimple portions, each dimple portion of the plurality of dimple portions having an increased valve body thickness, wherein the valve is collapsible relative to each of the plurality of dimple portions to permit fluid flow toward the opening of the connector.

Accordingly, the present application addresses several operational challenges encountered in prior valves, including needleless access valves, used in administering fluid using a syringe and provides numerous enhancements and improvements for the consistent collapse and restoration of the valve, which can reduce valve fold over, valve stick, and valve failure.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a perspective view of a connector, in accordance with some aspects of the present disclosure.

FIG. 2 illustrates the connector assembly in FIG. 1 in use with a fluid delivery device, in accordance with some aspects of the present disclosure.

FIGS. 3 and 4 illustrate partial cross-sectional views of the connector assembly, in accordance with some aspects of the present disclosure.

FIG. 5 is a cross-sectional view of the valve of the connector assembly depicted in FIGS. 3 and 4.

FIG. 6 illustrates a partial cross-sectional view of the connector assembly, further showing a fluid delivery device inserted into the housing and engaging the valve, in accordance with some aspects of the present disclosure.

FIG. 7 illustrates a partial cross-sectional view of the connector assembly, with the valve in a collapsed position.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Needle-free connectors are essential devices to deliver fluid to a patient via an IV catheter. Needle-free connectors may be used in general patient populations, including neonatal, pediatric, and adult patients. Certain conventional connectors may include valves that collapse in a random and/or inconsistent manner. In some applications, valves that collapse in an inconsistent manner may provide an inconsistent activation force. In certain applications, such as high pressure applications (e.g. power injection procedures), certain conventional valves may collapse and fold over on itself, eliminating a valve restoring force or otherwise remain stuck in an open position. A folded over or “stuck” valve may not return to normal operation without physical intervention or full replacement of the IV set. The following devices and methods provide design modifications to overcome the foregoing issues.

Embodiments described herein can provide a modified valve geometry to improve the functionality of a connector. Further, embodiments described herein can provide a valve that collapses in an intentional, consistent, and/or even manner can prevent valve failures, such as stuck or folded valves. Advantageously, embodiments of the valve can have bellows with cutouts and/or geometries (e.g. varying thicknesses and diameters) to allow for crumple zones and consistent collapse and restoration of the valve.

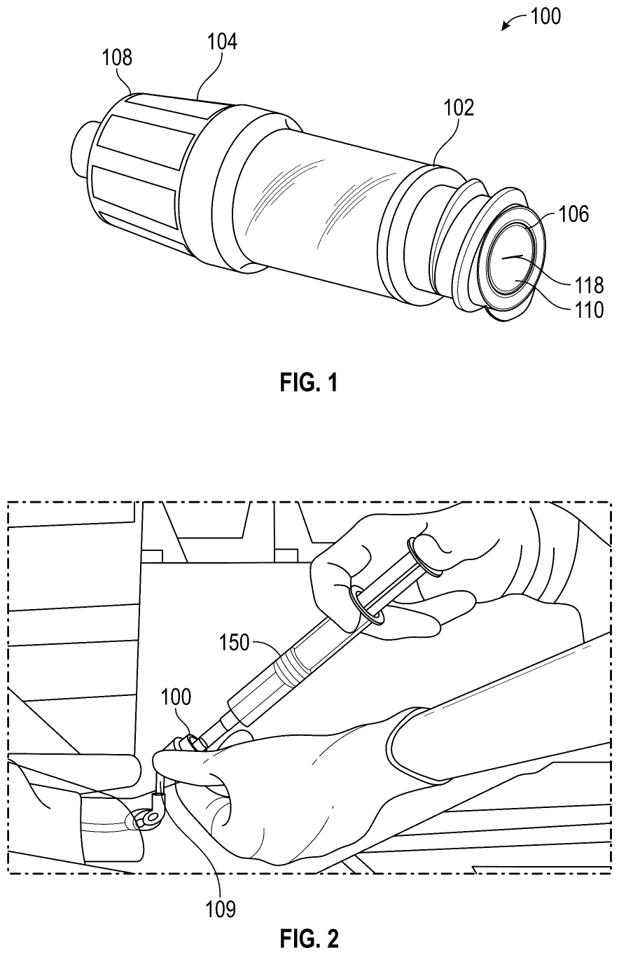

Referring now to the figures, FIGS. 1 and 2 illustrate a perspective view of a connector assembly 100, in accordance with some aspects of the present disclosure. The connector assembly 100 may include a housing 102 and a base 104 coupled with the housing 102. The housing 102 and the base 104 may be referred to as a first housing and a second housing, respectively. The housing 102 includes a fluid inlet 106, and the base 104 includes a fluid outlet 108. As shown, the fluid inlet 106 and the fluid outlet 108 are cylindrical, or generally cylindrical, bodies with circular cross sections. However, other shapes are possible. To regulate the fluid, the connector assembly 100 includes a valve 110 carried within portions of the housing 102 and the base 104.

As shown in FIG. 2, the connector assembly 100 may be used to provide a connection point for a fluid delivery device 150. In some embodiments, the fluid delivery device 150 includes a syringe, such as a needle-free syringe. Accordingly, in some embodiments, the connector assembly 100 can take the form of a needle-free connector assembly. For some exemplary IV applications, the fluid delivery device 150 may be used for rapid injections of medication, sometimes referred to as a “push” or “bolus” to quickly send a one-time dose of medication into a patient's bloodstream. The fluid delivery device 150 is connected to the fluid inlet 106 (shown in FIG. 1) of the connector assembly 100. Additionally, the fluid outlet 108 of the connector assembly 100 can be connected to a catheter line 109. The valve 110 is positioned to regulate the flow of fluid provided by the fluid delivery device 150 to the catheter line 109.

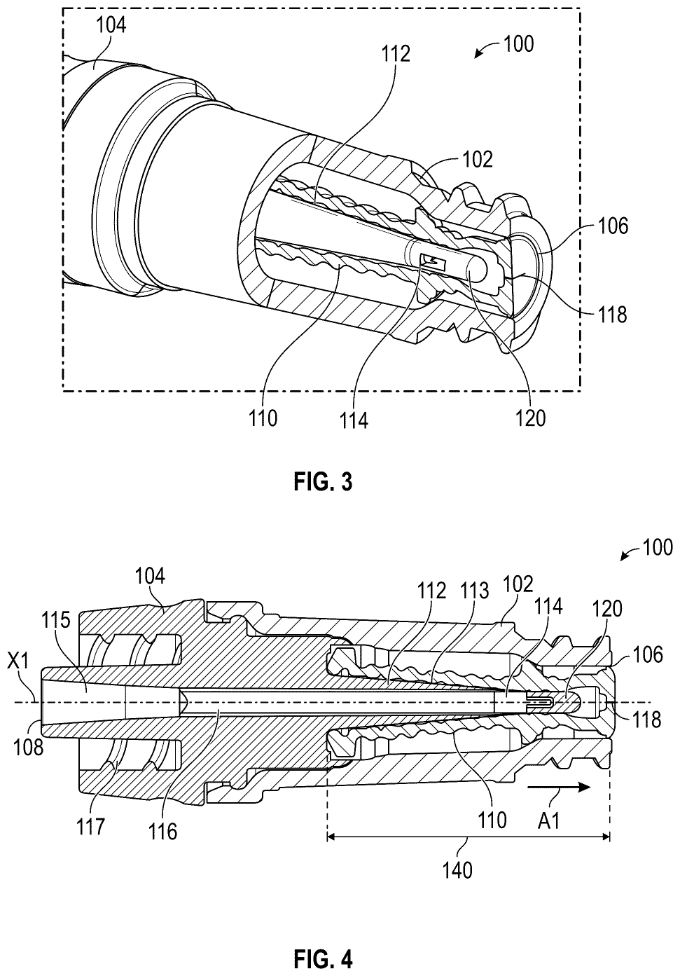

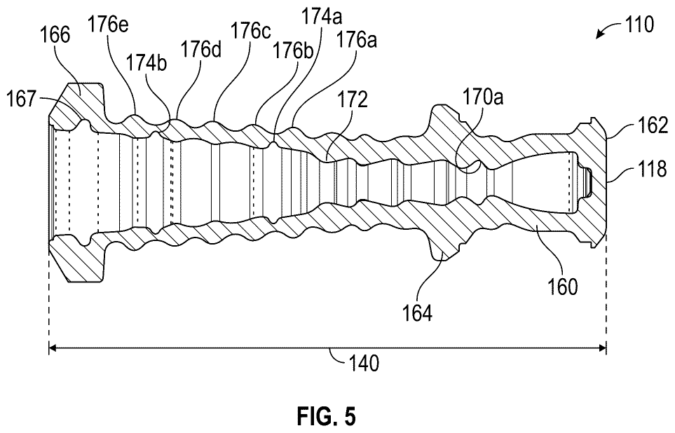

FIGS. 3 and 4 illustrate partial cross-sectional views of the connector assembly 100, in accordance with some aspects of the present disclosure. FIG. 5 is a cross-sectional view of the valve of the connector assembly depicted in FIGS. 3 and 4. The base 104 may include a post 112 that extends into the housing 102. The post 112 provides a hollow body to facilitate the transfer of fluid through the connector assembly 100. The post 112 includes a proximal end and a distal end 120, and, in some embodiments of the present disclosure, an outer surface of the post defines a cross-sectional width that decreases in a direction from the proximal end toward the distal end 120 of the post. In some embodiments, the post 112 can extend further or less into the housing 102 and/or relative to the valve 110. In some embodiments, the base 104 may not include a post.

The valve 110 includes a channel 113 that allows the valve 110 to receive the post 112. As shown in FIGS. 3 and 4, the valve 110 surrounds the post 112. In some embodiments, the valve 110 is disposed on, and engaged with, the post 112. As illustrated, the valve 110 can include one or more sealing portions 172 formed within the channel 113 to seal against the post 112. In some embodiments, the sealing portions 172 can be configured to minimize friction as the valve 110 moves relative to the post 112.

Also, the post 112 includes an opening 114 (representative of one or more openings in the post 112) designed to receive fluid from the fluid inlet 106 of the housing 102. For purposes of illustration, the post 112 in FIG. 4 is rotated such that the opening 114 is viewable. As shown in FIG. 4, a lumen 116 is formed by or positioned in a channel 115 of the post 112. The fluid, when received from the opening 114, can subsequently flow into the lumen 116. Accordingly, the opening 114 is fluidly connected to the lumen 116. In order to secure the lumen 116, the base 104 includes a luer 117.

Based on the position of the valve 110 shown in FIGS. 3 and 4, the valve 110 is in a closed position and covers the opening 114, thereby preventing the flow of fluid into the opening 114 of the post 112 and to the lumen 116. However, the valve 110 is designed to regulate flow based in part upon the displacement (e.g., compression) of the valve 110 such that the opening 114 is uncovered by the valve 110, as will be shown and described in detail below. It should be noted that displacement of the valve 110 may include an elastic displacement (e.g., elastic compression), thus allowing the valve 110 to return, after displacement, to its original form (shown in FIGS. 3 and 4) when a fluid delivery device is removed/disengaged from the valve 110.

In the depicted example, the valve 110 can have features, geometries, or otherwise be configured to provide consistent and even collapsing and avoid the valve body 160 from becoming stuck during operation (including high pressure operation). In some embodiments, the valve body 160 can have features or dimples 176 (dimples 176a-176e shown) that are shaped or arranged to allow for consistent collapse of the valve body 160 during operation. As illustrated, the dimples 176 can have a generally rounded shape and be formed on the exterior surface of the valve body 160. The dimples 176 can be arranged to allow for portions of the valve body 160 to have increased thickness and stiffness and other portions to have reduced thickness and stiffness, allowing the valve body 160 to collapse in a consistent and desired manner during operation.

In some embodiments, the valve body 160 can have relief portions 174 (relief portions 174a, 174b shown) that are also shaped or arranged to allow for consistent collapse of the valve body 160 during operation. As illustrated, the relief portions 174 can be grooves, channels, or voids formed in the interior surface of the lumen 116 within the valve 110. The relief portions 174 can be arranged to provide areas of reduced valve body 160 thickness and stiffness to introduce locations where the valve body 160 can consistently collapse.

In the depicted example, the dimples 176 and the relief portions 174 can be arranged to cooperatively provide areas of increased valve body 160 stiffness and areas of reduced valve body 160 stiffness to facilitate consistent collapse of the valve 110 during operation. For example, in some embodiments, a relief portion 174a can be disposed between a first dimple 176a and a second dimple 176b to allow the valve body 160 to collapse outward between the first dimple 176a and the second dimple 176b. Similarly, in some embodiments, a relief portion 174b can be disposed between a fourth dimple 176d and a fifth dimple 176e to allow the valve body 160 to collapse outward between the fourth dimple 176d and the fifth dimple 176e. In some embodiments, the third dimple 176c can be configured to collapse inward. In other words, in some embodiments, the valve body is configured in a “2-1-2” configuration, such that the first and second dimple 176a, 176b collapse outward, the third dimple 176c collapses inward, and the fourth and fifth dimple 176d, 176e collapse outward.

In some embodiments, other portions of the valve body 160 can include additional relief portions to facilitate a consistent collapse of the valve 110. For example, in some embodiments, the valve body 160 can include a relief portion 170 disposed adjacent to the mating portion 164 of the valve body 160. Similarly, in some embodiments, the valve body 160 can include a relief portion 167 disposed adjacent to the end portion 166 of the valve body 160. As described above, the relief portions 167, 170 can provide an area of reduced stiffness to facilitate the valve body 160 to collapse in a desired manner.

Further, in some embodiments, the valve body 160 can be formed to have increased body stiffness compared to certain conventional valves. As described herein, the valve body 160 can include one or more features that increases the stiffness of the valve body 160. Further, in some embodiments, the valve body 160 can be formed from a material with an increased, decreased, or otherwise different stiffness compared to certain conventional valves. The valve body 160 can be configured to provide a desired activation force and a desired restoration force.

Also, a slit 118, representing a cut or other discontinuity in the valve 110, is formed in the valve 110. In the closed position of the valve 110, no object(s) is/are positioned in the slit 118 of the valve 110 and the slit 118 is generally closed. For example, a distal end 120 of the post 112 does not protrude through the slit 118 in the closed position of the valve 110.

Referring to FIG. 4, a longitudinal axis X1 represents an axis that runs parallel to a major dimension of the connector assembly 100. The housing 102 of the connector assembly 100 includes a size such that when the housing 102 is coupled with the base 104 (as shown in FIG. 3), the housing 102 extends further along the longitudinal axis X1, in the direction of the arrow A1 of the longitudinal axis X1, than the post 112. Put another way, the distal end 120 of the post 112 is contained within the housing 102 and does not protrude out of the fluid inlet 106 of the housing 102. Also, when no external forces are acting on the valve 110 and the valve 110 is in the closed position, the valve 110 includes a dimension 140 that represents a lengthwise dimension of the valve 110.

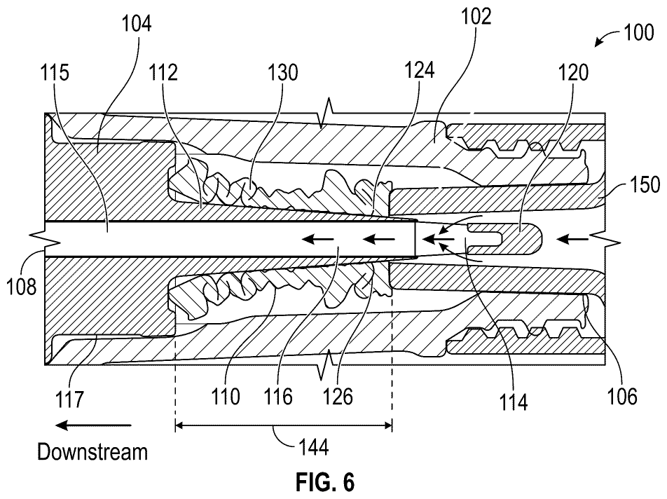

FIG. 6 illustrates a partial cross-sectional view of the connector assembly 100, further showing a fluid delivery device 150 inserted into the fluid inlet 106 of the housing 102 and engaging the valve 110, in accordance with some aspects of the present disclosure. The fluid inlet 106 of the housing 102 includes a size and shape that allows the fluid delivery device 150 to enter the housing 102 through the fluid inlet 106 and engage the valve 110. From the perspective of the valve 110, an external force is applied by the fluid delivery device 150. The external force provided by the fluid delivery device 150 causes displacement of the valve 110. For example, as shown in FIG. 6, the valve 110 compresses, or reduces to a dimension 144 (less than the dimension 140, shown in FIG. 4) based on the external force applied by the fluid delivery device 150. Also, the compression of the valve 110 represents relative movement of the valve 110 as compared to the housing 102, the base 104, and the post 112.

In the depicted example, the fluid delivery device 150 can engage with the mating portion 164 of the valve 110 to move or displace the valve body 160 In some embodiments, the fluid delivery device 150 can include features to engage with the mating portion 164 that prevent or restrain the outward radial movement of the valve body 160, minimizing undesired folding or sticking of the valve 110.

With reference to FIG. 6, based on the displacement of the valve 110, the valve 110 is in the open position and can receive fluid from the fluid delivery device 150. Several arrows are shown in the connector assembly 100 and the fluid delivery device 150. The arrows represent fluid flow from the fluid delivery device 150 and through the connector assembly 100. Also, as shown in FIG. 6, the downstream direction of fluid flow is shown. In the open position of the valve 110, the distal end 120 of the post 112 protrudes through the slit 118 of the valve 110. Additionally, the displacement of the valve 110 exposes the opening 114 of the post 112. For purposes of illustration, the post 112 in FIG. 6 is rotated such that the opening 114 is viewable. When the opening 114 is uncovered by the valve 10, fluid flows from the fluid delivery device 150 through the slit 118 (now open) and subsequently to the lumen 116 by way of the opening 114. Accordingly, the slit 118 is fluidly connected to the opening 114 and the lumen 116.

As a result of the displacement of the valve 110, a fluid pocket 124 is formed in the valve 110. The fluid pocket 124 represents a volume, i.e., three-dimensional cavity or recess in the valve 110. In some embodiments, during fluid delivery provided by the fluid delivery device 150, the fluid pocket 124 receives at least some of the fluid. The volume of the fluid pocket 124 is dependent upon the amount of displacement of the valve 110 caused by the external pressure from the fluid delivery device 150 and may vary, as described below.

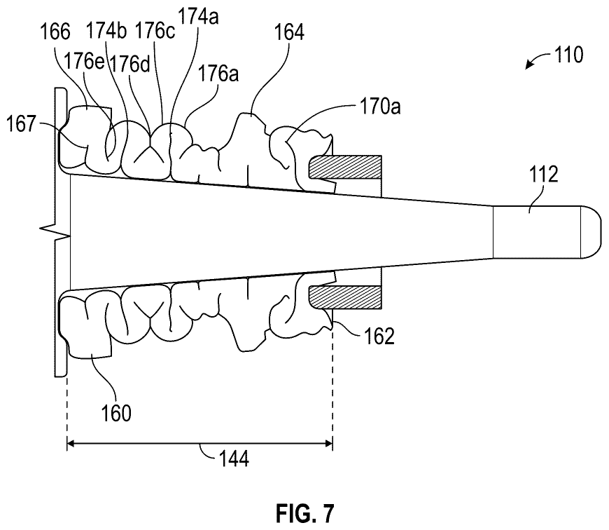

FIG. 7 illustrates a partial cross-sectional view of the connector assembly, with the valve in a collapsed position. Based on the increased insertion into the fluid inlet 106 of the housing 102, the fluid delivery device 150 applies an additional external force to the valve 110, causing further displacement of the valve 110. For example, the valve 110 is further compressed within the housing 102, causing the distal end 120 of the post 112 to further protrude through the slit 118 of the valve 110.

As described herein, the valve 110 can have features, geometries, or otherwise be configured to collapse to the reduced dimension 144 in a consistent and even manner to avoid folding over or otherwise becoming stuck. As illustrated in FIG. 7, portions of the valve body 160 can be configured to consistently collapse radially outward during operation (i.e. compression) and other portions of the valve body 160 can be configured to consistently collapse radially inward during operation to allow for the collapse and restoration of the valve 110 during operation.

As illustrated, the portion of the valve body 160 including the first dimple 176a and the second dimple 176b can collapse radially outward relative to the post 112. As described herein, the first relief portion 174a can further facilitate the radial outward collapse of this portion of the valve body 160. In some embodiments, the portion of the valve body 160 including the third dimple 176c can collapse radially inward relative to the post 112. Similarly, the portion of the valve body 160 including the fourth dimple 176d and the fifth dimple 176e can collapse radially outward relative to the post 112. As described herein, the second relief portion 174b can further facilitate the radial outward collapse of this portion of the valve body 160. In the depicted example, the valve 110 can consistently collapse into the depicted “2-1-2” configuration to allow for consistent collapse and restoration of the valve 110.

In some embodiments, other portions of the valve body 160 can collapse and restore in a consistent manner. For example, the mating portion 164 of the valve body 160 can collapse radially outward relative to the post 112. A relief portion 170 can facilitate the radial outward collapse of the mating portion 164. Similarly, the end portion 166 of the valve body 160 can collapse radially outward relative to the post 112. The relief portion 167 can facilitate the radial outward collapse of the end portion 166.

As the fluid delivery device 150 (previously shown) is removed from the housing 102, the valve 110 begins to decompress. For example, the distal end 120 of the post 112 no longer protrudes through the slit 118 of the valve 110, and the valve 110 returns to the closed position. As a result, the valve 110 prevents fluid (external to the valve 110) from subsequently entering the valve 110. Accordingly, the valve 110 covers the opening 114 and the distal end 120 of the post 112. When the fluid delivery device 150 is removed from the fluid inlet 106 of the housing 102 and no longer engages the valve 110, the valve 110 is no longer displaced and returns to its original shape (shown in FIGS. 3 and 4). Although not shown, the valve 110 may cover the opening 114 prior to the fluid delivery device 150 being fully removed from the fluid inlet 106 of the housing 102 and prior to the fluid delivery device 150 being fully disengaged from the valve 110. As described herein, the features, geometries, and/or configuration of the valve 110 allows the valve 110 to restore to its original shape without becoming stuck or requiring intervention.

Although the present disclosure includes embodiments in which a post includes a single opening in the Figures, it should be understood that the post may include any number of openings, each of each can receive a fluid from a fluid delivery device.

The features of the present disclosure provide multiple housings can be coupled together to form a fluid pathway therebetween. When coupled together, the features of the present disclosure resist unintentional separation between the housings.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A connector assembly for use with a catheter, the connector assembly including: a first housing including a fluid inlet; a second housing coupled with the first housing, the second housing including a post defining an opening; and a valve including a channel disposed around the post to selectively isolate the opening from the fluid inlet, the channel defining a relief portion having a reduced valve body thickness, wherein the valve is collapsible relative to the relief portion to permit fluid communication between the fluid inlet and the opening.

Clause 2. The connector assembly of Clause 1, the valve including an exterior surface defining a dimple portion having an increased valve body thickness, wherein the valve is collapsible relative to the dimple portion and the relief portion.

Clause 3. The connector assembly of Clause 2, wherein the exterior surface includes a second dimple portion, wherein the relief portion is disposed between the dimple portion and the second dimple portion.

Clause 4. The connector assembly of Clause 3, wherein the dimple portion and the second dimple portion are collapsible radially outward.

Clause 5. The connector assembly of Clause 3, wherein the channel defines a second relief portion, wherein the valve is collapsible relative to the relief portion and the second relief portion.

Clause 6. The connector assembly of Clause 5, wherein the exterior surface includes a third dimple portion and a fourth dimple portion, wherein the second relief portion is disposed between the third dimple portion and the fourth dimple portion.

Clause 7. The connector assembly of Clause 6, wherein the third dimple portion and the fourth dimple portion are collapsible radially outward.

Clause 8. The connector assembly of Clause 6, wherein the exterior surface includes a fifth dimple portion disposed between the second dimple portion and the third dimple portion.

Clause 9. The connector assembly of Clause 8, wherein the fifth dimple portion is collapsible radially inward.

Clause 10. The connector assembly of Clause 9, wherein the dimple portion and the second dimple portion are collapsible radially outward and the third dimple portion and the fourth dimple portion are collapsible radially outward.

Clause 11. A connector assembly for use with a catheter, the connector assembly including: a first housing including a fluid inlet; a second housing coupled with the first housing, the second housing including a post defining an opening; and a valve including an exterior surface defining a dimple portion having an increased valve body thickness, the valve disposed around the post to selectively isolate the opening from the fluid inlet, wherein the valve is collapsible relative to the dimple portion to permit fluid communication between the fluid inlet and the opening.

Clause 12. The connector assembly of Clause 11, wherein the exterior surface includes a second dimple portion.

Clause 13. The connector assembly of Clause 12, wherein the dimple portion and the second dimple portion are collapsible radially outward.

Clause 14. The connector assembly of Clause 12, wherein the exterior surface includes a third dimple portion and a fourth dimple portion.

Clause 15. The connector assembly of Clause 14, wherein the third dimple portion and the fourth dimple portion are collapsible radially outward.

Clause 16. The connector assembly of Clause 14, wherein the exterior surface includes a fifth dimple portion disposed between the second dimple portion and the third dimple portion.

Clause 17. The connector assembly of Clause 16, wherein the dimple portion and the second dimple portion are collapsible radially outward and the third dimple portion and the fourth dimple portion are collapsible radially outward.

Clause 18. A collapsible valve for use with a connector, the valve including: a channel configured to selectively isolate to an opening of the connector; and an exterior surface defining a plurality of dimple portions, each dimple portion of the plurality of dimple portions having an increased valve body thickness, wherein the valve is collapsible relative to each of the plurality of dimple portions to permit fluid flow toward the opening of the connector.

Clause 19. The collapsible valve of Clause 18, wherein the plurality of dimples includes: a first dimple portion and a second dimple portion, wherein the first dimple portion and the second dimple portion are collapsible radially outward; a third dimple portion and a fourth dimple portion, wherein the third dimple portion and the fourth dimple portion are collapsible radially outward; and a fifth dimple portion disposed between the second dimple portion and the third dimple portion, wherein the fifth dimple portion is collapsible radially inward.

Clause 20. The collapsible valve of Clause 19, wherein the channel defines a first relief portion and a second relief portion, the first relief portion and the second relief portion having a reduced valve body thickness, the first relief portion disposed between the first dimple portion and the second dimple portion, and the second relief portion disposed between the third dimple portion and the fourth dimple portion.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A connector assembly for use with a catheter, the connector assembly comprising:
a first housing comprising a fluid inlet;
a second housing coupled with the first housing, the second housing comprising a post defining an opening; and
a valve comprising a channel disposed around the post to selectively isolate the opening from the fluid inlet, the channel defining a relief portion having a reduced valve body thickness, wherein the valve is collapsible relative to the relief portion to permit fluid communication between the fluid inlet and the opening.

2. The connector assembly of claim 1, the valve comprising an exterior surface defining a dimple portion having an increased valve body thickness, wherein the valve is collapsible relative to the dimple portion and the relief portion.

3. The connector assembly of claim 2, wherein the exterior surface comprises a second dimple portion, wherein the relief portion is disposed between the dimple portion and the second dimple portion.

4. The connector assembly of claim 3, wherein the dimple portion and the second dimple portion are collapsible radially outward.

5. The connector assembly of claim 3, wherein the channel defines a second relief portion, wherein the valve is collapsible relative to the relief portion and the second relief portion.

6. The connector assembly of claim 5, wherein the exterior surface comprises a third dimple portion and a fourth dimple portion, wherein the second relief portion is disposed between the third dimple portion and the fourth dimple portion.

7. The connector assembly of claim 6, wherein the third dimple portion and the fourth dimple portion are collapsible radially outward.

8. The connector assembly of claim 6, wherein the exterior surface comprises a fifth dimple portion disposed between the second dimple portion and the third dimple portion.

9. The connector assembly of claim 8, wherein the fifth dimple portion is collapsible radially inward.

10. The connector assembly of claim 9, wherein the dimple portion and the second dimple portion are collapsible radially outward and the third dimple portion and the fourth dimple portion are collapsible radially outward.

11. A connector assembly for use with a catheter, the connector assembly comprising:
a first housing comprising a fluid inlet;
a second housing coupled with the first housing, the second housing comprising a post defining an opening; and
a valve comprising an exterior surface defining a dimple portion having an increased valve body thickness, the valve disposed around the post to selectively isolate the opening from the fluid inlet, wherein the valve is collapsible relative to the dimple portion to permit fluid communication between the fluid inlet and the opening.

12. The connector assembly of claim 11, wherein the exterior surface comprises a second dimple portion.

13. The connector assembly of claim 12, wherein the dimple portion and the second dimple portion are collapsible radially outward.

14. The connector assembly of claim 12, wherein the exterior surface comprises a third dimple portion and a fourth dimple portion.

15. The connector assembly of claim 14, wherein the third dimple portion and the fourth dimple portion are collapsible radially outward.

16. The connector assembly of claim 14, wherein the exterior surface comprises a fifth dimple portion disposed between the second dimple portion and the third dimple portion.

17. The connector assembly of claim 16, wherein the dimple portion and the second dimple portion are collapsible radially outward and the third dimple portion and the fourth dimple portion are collapsible radially outward.

18. A collapsible valve for use with a connector, the valve comprising:
a channel configured to selectively isolate to an opening of the connector; and
an exterior surface defining a plurality of dimple portions, each dimple portion of the plurality of dimple portions having an increased valve body thickness, wherein the valve is collapsible relative to each of the plurality of dimple portions to permit fluid flow toward the opening of the connector.

19. The collapsible valve of claim 18, wherein the plurality of dimples comprises:
a first dimple portion and a second dimple portion, wherein the first dimple portion and the second dimple portion are collapsible radially outward;
a third dimple portion and a fourth dimple portion, wherein the third dimple portion and the fourth dimple portion are collapsible radially outward; and
a fifth dimple portion disposed between the second dimple portion and the third dimple portion, wherein the fifth dimple portion is collapsible radially inward.

20. The collapsible valve of claim 19, wherein the channel defines a first relief portion and a second relief portion, the first relief portion and the second relief portion having a reduced valve body thickness, the first relief portion disposed between the first dimple portion and the second dimple portion, and the second relief portion disposed between the third dimple portion and the fourth dimple portion.
